(19) United States
(12) Patent Application Publication
(10) Pub. No.: US 2020/0090818 A1
(43) Pub. Date: Mar. 19, 2020

(54) SYSTEM AND METHOD FOR MAPPING BIOMEDICAL ENTITIES

(71) Applicant: Innoplexus AG, Eschborn (DE)

(72) Inventors: Gaurav Tripathi, Pune (IN); Om Sharma, Dhanbad (IN); Vatsal Agarwal, Rampur (IN); Anurag Chandrekar, Raipur (IN)

(21) Appl. No.: 16/366,451

(22) Filed: Mar. 27, 2019

(30) Foreign Application Priority Data
Mar. 27, 2018 (GB) 1804894.2

(51) Int. Cl. G16H 70/60; G16H 70/40; G16H 50/70; G06F 16/901

(52) U.S. Cl. CPC G16H 70/60; G06F 16/901; G16H 50/70; G16H 70/40

(57) ABSTRACT

A system and method of mapping biomedical entities, wherein each of the biomedical entities belong to one of a predefined class: target, disease, pathway, and drug. The system includes a database arrangement operable to store existing data sources; a processing module communicably coupled to the database arrangement. The processing module operable to receive a user-input of a biomedical entity belonging to one of the predefined classes, wherein the predefined class of the biomedical entity defines an input class; extract a plurality of biomedical entities related to the biomedical entity of the user-input from existing data sources, wherein the plurality of biomedical entities belong to predefined classes except the input class; identify at least one pair of biomedical entities, from the plurality of extracted biomedical entities, having an association therebetween; and map the plurality of biomedical entities to the biomedical entity of the user-input.

### CROSS-REFERENCE TO RELATED APPLICATIONS

This application claims the benefit under 35 U.S.C. § 119(a) and 37 CFR § 1.55 to UK Patent Application No. GB1804894.2, filed on Mar. 27, 2018, the entire content of which is incorporated herein by reference.

### TECHNICAL FIELD

The present disclosure relates generally to data processing; and more specifically, to bioinformatics. Furthermore, the present disclosure also relates to systems that maps associations between biomedical entities. Moreover, the present disclosure relates to methods for mapping associations between biomedical entities. Moreover, the present disclosure also relates to computer readable medium containing program instructions for execution on a computer system, which when executed by a computer, cause the computer to perform method steps for mapping biomedical entities.

### BACKGROUND

In recent years, increased population and pollution have led to an unprecedented growth of diseases across the globe. In order to overcome the array of challenges, drug discoveries are advancing rapidly, researches and experiments are also going on a regular basis. With the advent of technology and breakthrough researches in field of medicines, a number of medications and treatment are available for treating health ailments. Generally, researchers and physicians study several biomedical entities relating to human anatomy and pharmaceutical compounds. Furthermore, associations between such biomedical entities provides invaluable insights relating to diseases and treatments.

Furthermore, due to a rapid increase in the number of research publications, clinical trials, research related to such biomedical entities using relevant existing information from has become challenging. Furthermore, currently available techniques access information regarding such biomedical entities from Internet® or several other existing data sources. However, such data sources may lack in information and may not comprise relevant updated information. Furthermore, a user may need to access several data sources in order to retrieve relevant information related to the biomedical entities. Therefore, the currently available techniques for accessing data may require extensive input from the user. Additionally, the conventional techniques provide ambiguous information related to the biomedical entities and associations therebetween. Consequently, the currently available techniques for accessing information regarding medication, disease, and other factors associated thereto do not provide optimal, adequate and centralized information.

Therefore, in light of the foregoing discussion, there exists a need to overcome the aforementioned drawbacks associated with accessing data related to biomedical entities.

### SUMMARY

The present disclosure seeks to provide a system that maps the biomedical entities, wherein each of the biomedical entities belongs to one of a predefined class: target, disease, pathway, drug. The present disclosure also seeks to provide a method of mapping biomedical entities. The present disclosure also seeks to provide a computer readable medium, containing program instructions for execution on a computer system, which when executed by a computer, cause the computer to perform method steps for mapping biomedical entities. Furthermore, the present disclosure seeks to provide a solution to the existing problem of redundant, unorganized and unmanageable biomedical data. Moreover, the present disclosure provides an optimal way of substantially reducing effort required in accessing biomedical data. An aim of the present disclosure is to provide a solution that overcomes at least partially the problems encountered in prior art, and provides an efficient method and system for mapping biological entities

In one aspect, an embodiment of the present disclosure provides a system that maps biomedical entities, wherein each of the biomedical entities belongs to one of a predefined class: target, disease, pathway, and drug, wherein the system includes a computer system, wherein the system comprises:
a database arrangement operable to store existing data sources; and
a processing module communicably coupled to the database arrangement, the processing module operable to:
   receive a user-input of a biomedical entity belonging to one of the predefined classes, wherein the predefined class of the biomedical entity defines an input class;
   extract a plurality of biomedical entities related to the biomedical entity of the user-input from existing data sources, wherein the plurality of biomedical entities belongs to predefined classes except the input class;
   identify at least one pair of biomedical entities, from the plurality of extracted biomedical entities, having an association therebetween, wherein each biomedical entity of the at least one pair of biomedical entities belongs to different predefined classes; and
   map the plurality of biomedical entities to the biomedical entity of the user-input.

In another aspect, an embodiment of the present disclosure provides a method of mapping biomedical entities, wherein each of the biomedical entities belongs to one of a predefined class: target, disease, pathway, and drug, wherein the method includes using a computer system, wherein the method comprises:
providing a user-input of a biomedical entity belonging to one of the predefined classes, wherein the predefined class of the biomedical entity defines an input class;
extracting a plurality of biomedical entities related to the biomedical entity of the user-input from existing data sources, wherein the plurality of biomedical entities belongs to predefined classes except the input class;
identifying at least one pair of biomedical entities, from the plurality of extracted biomedical entities, having an association therebetween, wherein each biomedical entity of the at least one pair of biomedical entities belongs to different predefined classes; and
mapping the plurality of biomedical entities to the biomedical entity of the user-input.

In yet another aspect, an embodiment of the present disclosure provides a computer readable medium, containing program instructions for execution on a computer system, which when executed by a computer, cause the computer to perform method steps for mapping biomedical entities, wherein each of the biomedical entities belongs to one of a predefined class: target, disease, pathway, and drug, the method comprising the steps of:
providing a user-input of a biomedical entity belonging to one of the predefined classes, wherein the predefined class of the biomedical entity defines an input class;
extracting a plurality of biomedical entities related to the biomedical entity of the user-input from existing data sources, wherein the plurality of biomedical entities belongs to predefined classes except the input class;
identifying at least one pair of biomedical entities, from the plurality of extracted biomedical entities, having an association therebetween, wherein each biomedical entity of the at least one pair of biomedical entities belongs to different predefined classes; and
mapping the plurality of biomedical entities to the biomedical entity of the user-input.

Embodiments of the present disclosure substantially eliminate or at least partially address the aforementioned problems in the prior art, and enables an efficient, effective, seamless, structured and optimal method of mapping associations among biomedical entities including: target, disease, pathway, drug.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1 is illustration of steps of a method of mapping biomedical entities, in accordance with an embodiment of the present disclosure;

FIG. 2 is a block diagram of a system that maps biomedical entities, in accordance with an embodiment of the present disclosure; and

FIGS. 3-4 are schematic illustrations of exemplary implementations of the method of mapping biomedical entities, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

In overview, embodiments of the present disclosure are concerned with mapping biomedical entities and specifically to, determining associations of a given biomedical entity in a pharmaceutical network.

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In one aspect, an embodiment of the present disclosure provides a system that maps biomedical entities, wherein each of the biomedical entities belongs to one of a predefined class: target, disease, pathway, and drug, wherein the system includes a computer system, wherein the system comprises:
a database arrangement operable to store existing data sources; and
a processing module communicably coupled to the database arrangement, the processing module operable to:
   receive a user-input of a biomedical entity belonging to one of the predefined classes, wherein the predefined class of the biomedical entity defines an input class;
   extract a plurality of biomedical entities related to the biomedical entity of the user-input from existing data sources, wherein the plurality of biomedical entities belongs to predefined classes except the input class;
   identify at least one pair of biomedical entities, from the plurality of extracted biomedical entities, having an association therebetween, wherein each biomedical entity of the at least one pair of biomedical entities belongs to different predefined classes; and
   map the plurality of biomedical entities to the biomedical entity of the user-input.

In another aspect, an embodiment of the present disclosure provides a method of mapping biomedical entities, wherein each of the biomedical entities belong to one of a predefined class: target, disease, pathway, and drug, wherein the method includes using a computer system, wherein the method comprises:
providing a user-input of a biomedical entity belonging to one of the predefined classes, wherein the predefined class of the biomedical entity defines an input class;
extracting a plurality of biomedical entities related to the biomedical entity of the user-input from existing data sources, wherein the plurality of biomedical entities belongs to predefined classes except the input class;
identifying at least one pair of biomedical entities, from the plurality of extracted biomedical entities, having an association therebetween, wherein each biomedical entity of the at least one pair of biomedical entities belongs to different predefined classes; and
mapping the plurality of biomedical entities to the biomedical entity of the user-input.

The present disclosure provides the aforementioned system and method for mapping biomedical entities. The described method maps associations among biomedical entities including: target, disease, pathway, and drug. Thus, the method provides relevant and accurate associations between a pair of biomedical entities. The described method does not require users to exert manual effort in accessing biomedical entities associated with the biomedical entity of user-input. Consequently, the present disclosure provides an effortless and less time-consuming solution for retrieving biomedical data. Furthermore, the method provides a common platform for accessing relevant biomedical data associated to biomedical entity of user-input.

The computer system relates to at least one computing unit comprising a central storage system, processing units and various peripheral devices. Optionally, the computer system relates to an arrangement of interconnected computing units, wherein each computing unit in the computer system operates independently and may communicate with other external devices and other computing units in the computer system.

The term “system that maps” is used interchangeably with the term “system for mapping”, wherever appropriate i.e. whenever one such term is used it also encompasses the other term.

Throughout the present disclosure, the term “biomedical entities” refers to a therapeutic data unit related to biomedical sciences. Furthermore, the biomedical entities have an association therebetween based on functional aspect thereof. For example, the biomedical entity “Nexium” may be used to reduce production of stomach acid in human body, wherein “stomach acid” may be another biomedical entity. Furthermore, the biomedical entities and associations thereof are analysed to determine diagnosis, monitoring and therapy of a specific disease associated thereto. Additionally, the biomedical entities are mapped with related one or more biomedical entities in order to identify associations therebetween.

Furthermore, the term “mapping” used herein refers to determination of direct or indirect association (namely, relationship) between two or more biomedical entities. Furthermore, mapping is performed among biomedical entities having different features. Beneficially, mapping of biomedical entities provides non-ambiguous and non-redundant determination and representation of associations between biomedical entities. Furthermore, each of the biomedical entities belongs to one of a predefined class: target, disease, pathway and drug.

Throughout the present disclosure, the term “predefined class” refers to a specific group of entities having similar characteristics. Furthermore, the biomedical entities in one of the predefined class may have an association with one or more biomedical entities associated with any of the other predefined classes. For example, an anti-allergy medicine “cetirizine” may belong to predefined class: drug. Furthermore, the anti-allergy medicine “cetirizine” may be associated with a biomedical entity “allergy” belonging to predefined class: disease.

Moreover, biomedical entities belonging to the predefined class: target, are mainly enzymes and/or proteins. Specifically, the biomedical entities belonging to the predefined class: target, are biological sites generally associated with target sites for pharmaceutical drugs. More specifically, malfunctioning or anomaly in working of such biological site may cause a disease in a body (for example, a human body). In an example, “epidermal growth factor receptor” may be a protein belonging to the predefined class: target. Furthermore, a biomedical entity belonging to the predefined class: target has an association with one or more biomedical entities belonging to the predefined classes: disease, pathway and drug.

Furthermore, the predefined class: disease, includes biomedical entities with properties that cause adverse physiological effects on a subject (such as a human, animal and the like). The biomedical entities belonging to the predefined class: disease, have associations with one or more biomedical entities of the predefined classes: target, drug and pathway. In an example, a biomedical entity “Jaundice” belonging to the predefine class: disease, may have associations with biomedical entities “Bilirubin” belonging to the predefined class: target, and “phenobarbital” belonging to the predefined class: drug. Additionally, a biomedical entity belonging to the predefined class: disease, has associations with one or more biomedical entity belonging to the predefined class: pathway.

Moreover, the predefined class: pathway, relates to a collection (namely, series, route) of molecular regulators, chemical reactions, series of molecular events and so forth that lead to a certain product or physiological changes in the subject. Subsequently, such changes affect one or more biomedical entities, belonging to the predefined class: target, causing another biomedical entity belonging to the predefined class: disease.

Additionally, the predefined class: drug, includes biomedical entities such as a medicine, chemical compound, substance and the like that has a physiological effect when ingested, injected or otherwise introduced into the body. Furthermore, biomedical entities that belong to the predefined class: drug, have properties affecting a biomedical entity belonging to the predefined class: target. Additionally, such an affect reaches the biomedical entity belonging to the predefined class: target, through a biomedical entity belonging to the predefined class: pathway. Subsequently, such affect results in curing yet another specific biomedical entity belonging to the predefined class: disease.

As mentioned previously, the method of mapping biomedical entities comprises providing the user-input of the biomedical entity belonging to one of the predefined classes, wherein the predefined class of the biomedical entity defines the input class. Specifically, the processing module is operable to receive the user-input of the biomedical entity belonging to one of the predefined classes, wherein the predefined class of the biomedical entity defines the input class. Furthermore, the user-input is a biomedical entity belonging to any one of the predefined class: target, disease, pathway and drug. Additionally, the predefined class associated with the user-input biomedical entity is the input class. Specifically, at an instance the input class may be any one of the predefined class: target, disease, pathway and drug. In an example, the user input may be a biomedical entity “breast neoplasms”. Moreover, in the example, the biomedical entity of the user input belongs to the predefined class: disease. Therefore, in such example the input class is defined as the predefined class: disease. Moreover, the processing module is configured to receive the user-input using a user interface, drop down menu, command prompt and so forth.

Furthermore, the term “processing module” as used herein, relates to a computational element that is operable to respond to and process instructions. Optionally, the processing module includes, but is not limited to, a microprocessor, a microcontroller, a complex instruction set computing (CISC) microprocessor, a reduced instruction set (RISC) microprocessor, a very long instruction word (VLIW) microprocessor, or any other type of processing circuit. Furthermore, the term “processing module” may refer to one or more individual processors, processing devices and various elements associated with a processing device that may be shared by other processing devices. Additionally, the one or more individual processors, processing devices and elements are arranged in various architectures for responding to and processing the instructions.

Therefore, the processing module is communicably coupled to the database arrangement. Furthermore, the database arrangement is operable to store the existing data sources. Additionally, the term “existing data sources” as used herein, relates to organized or unorganized sources of digital information regardless of the manner in which the information is represented therein. Specifically, such digital information is related to biomedical entities namely, target, disease, pathway and drug. Furthermore, the existing data sources may be publicly available internet sources. For example, the existing data sources may include research publications, clinical trials, company websites, blogs, news websites, research institute websites, government websites, online surveys and so forth. Additionally, the existing data sources include biomedical entities and associated information thereof related to previous user-input of biomedical entity.

The processing module is communicably coupled to the database arrangement. Furthermore, the processing module may be coupled to the database arrangement using a network. Moreover, the network may relate to an arrangement of interconnected programmable and/or non-programmable components that are configured to facilitate data communication between one or more electronic devices, software modules and/or databases, whether available or known at the time of filing or as later developed. Additionally, the network employs wired or wireless communication that can be carried out via any number of known protocols.

As mentioned previously, the method of mapping biomedical entities further comprises extracting the plurality of biomedical entities related to the biomedical entity of the user-input from existing data sources, wherein the plurality of biomedical entities, belong to predefined classes except the input class. Specifically, the processing module is operable to extract the plurality of biomedical entities related to the biomedical entity of the user-input from existing data sources, wherein the plurality of biomedical entities, belong to predefined classes except the input class. Furthermore, biomedical entities associated to the user-input of the biomedical entity belong to one of the predefined class. Subsequently, the plurality of biomedical entities related to the biomedical entity of the user-input belongs to any of the predefined classes except the predefined class identical to the input class. In an instance when the input class is drug, the extracted plurality of biomedical entities may belong to the predefined class: target, disease and pathway. In an example, when user-input is a biomedical entity “EGFR” belonging to the predefined class: target. Consequently, the predefined class: target is defined as the input class. Additionally, plurality of biomedical entities may be extracted, wherein the plurality of biomedical entities belongs to the predefined classes except for the input class namely, disease, pathway and drug. The extracted plurality of biomedical entities may include “MAPK Signalling” belonging to predefined class: pathway, “Lung Neoplasm” belonging to predefined class: disease and “Trastuzumab” belonging to the predefined class: drug.

Optionally, when input class is predefined class: drug, each of the extracted plurality of biomedical entities may belong to one of the predefined classes: target, disease and pathway. More optionally, when input class is predefined class: disease, each of the extracted plurality of biomedical entities may belong to one of the predefined classes: target, drug and pathway. Additionally, when input class is predefined class: pathway, each of the extracted plurality of biomedical entities may belong to one of the predefined classes: target, disease and drug.

Optionally, the method further comprises tagging the biomedical entities in the predefined class: drug, with one of the tags: approved, investigational, combinational, and potential. Furthermore, the processing module is operable to tag the biomedical entities in the predefined class: drug, with one of the tags: approved, investigational, combinational, and potential. Specifically, tagging the biomedical entities in the predefined class drug provides additional information associated with a specific biomedical entity. Beneficially, such tagging may provide verified information associated with specific drugs. Moreover, tagging the biomedical entities in the predefined class drug with the tag “approved” indicates that the drug may be well known and frequently used in treatment of disease associated thereto. In an example, “Trastuzumab”, that is a known drug for treating “breast neoplasm”, may be tagged with the tag approved. Additionally, tagging the biomedical entities in the predefined class drug with the tag “investigational” indicates that the tagged biomedical entities may be under clinical trials. Consequently, effects of such biomedical entities may be uncertain. In another example, “BNC-105”, that is a drug in clinical trial for treatment of “breast neoplasm”, may be tagged with the tag “investigational”. Furthermore, tagging the biomedical entities in the predefined class drug with the tag “combinational” indicates that the tagged biomedical entities may have two or more active pharmaceutical ingredients combined in a single dosage form, which may be manufactured and distributed in fixed doses. In yet another example, “Ceftriaxone+Levofloxacin” may be two active pharmaceutical ingredients used in appropriate ration for treating “breast neoplasm”, may be tagged with the tag “combinational”. Furthermore, tagging the biomedical entities in the predefined class drug with the tag “potential” indicates that the tagged biomedical entities are approved drugs for one or more other biomedical entities belonging to the predefined class disease. In an example, “Viagra”, a drug used to treat erectile dysfunction. Consequently, such drug may be tagged “approved” for the biomedical entity: “erectile dysfunction”. However, “Viagra” may have a potential of use in treating pulmonary arterial hypertension, and thus, may be tagged with the tag “potential” for the biomedical entity: “Pulmonary Arterial Hypertension”. Consequently, tagging the biomedical entities belonging to the predefined class drug provides an efficient and informed way of identifying biomedical entities. Furthermore, it will be appreciated that the tag associated with a biomedical entity in the class: drug, may be determined in respect to another biomedical entity. In an example, a biomedical entity such as “Aspirin” may be tagged with the tag “approved” in respect to the biomedical entity: “Fever”. However, “Aspirin” may be tagged with a tag “potential” in respect to biomedical entity: “Gastrointestinal bleeding”.

The method further comprises identifying at least one pair of biomedical entities, from the plurality of extracted biomedical entities, having an association therebetween, wherein each biomedical entity of the at least one pair of biomedical entities belongs to different predefined classes. Specifically, the processing module is operable to identify at least one pair of biomedical entities, from the plurality of extracted biomedical entities, having an association therebetween, wherein each biomedical entity of the at least one pair of biomedical entities belongs to different predefined classes. Furthermore, characteristics of the extracted biomedical entities belonging to each of the predefined classes are analysed. Subsequently, biomedical entities, in the plurality of biomedical entities, with related functions and affects are paired in order to establish association therebetween. Additionally, biomedical entities in the at least one pair have a relation therebetween. Furthermore, the at least one identified pair of biomedical entities may have a direct or indirect relation with the biomedical entity of the user-input. In an instance when biomedical entity of the user-input may belong to the input class: target, a pair of biomedical entities belonging to the different predefined classes: disease and drug may be identified. In such an instance, the biomedical entity in the predefined class: disease may have a direct association with the biomedical entity of the user-input and the biomedical entity in the predefined class: drug might have an association with the biomedical entity of user-input through the biomedical entity in the predefined class: disease.

Optionally, the at least one pair of biomedical entities having the association therebetween comprises biomedical entities belonging to at least one of the following different predefined classes: target and drug, drug and disease, target and disease, pathway and disease, target and pathway. Specifically, in an instance when biomedical entities in the pair belong to the predefined classes: target and drug respectively, the biomedical entity belonging to the predefined class drug influences performance of the biomedical entity belonging to the predefined class target. Additionally, such an association might have a further association with another biomedical entity belonging to predefined class: disease. In a first example, a biomedical entity “EGFR” belonging to the predefined class: target may have an association with another biomedical entity “Trastuzumab” belonging to the predefined class: drug. Specifically, “Trastuzumab” influences EGFR levels in the cancer cells. In another instance, when biomedical entities in the pair belong to the predefined classes: drug and disease respectively, the biomedical entity belonging to the predefined class: drug may be used in treatment of the biomedical entity belonging to the predefined class: disease. Referring to the first example, the biomedical entity “Trastuzumab” belonging to the predefined class: drug may have an association with another biomedical entity “breast neoplasm” belonging to the predefined class: disease. The drug “Trastuzumab” may be used in treatment of the disease “breast neoplasm”. In yet another instance, when biomedical entities in the pair belong to the predefined classes: target and disease respectively, the biomedical entity belonging to the predefined class: disease may be caused because of abnormal functioning of the biomedical entity belonging to the predefined class: target. Furthermore, the biomedical entity belonging to the predefined class: disease may cause abnormal functioning of the biomedical entity belonging to the predefined class: target. Referring to the first example, the biomedical entity “EGFR” belonging to the predefined class: target may have an association with the biomedical entity “breast neoplasm” belonging to the predefined class: disease. Specifically, “breast neoplasm” associates with amplification (namely, overexpression) of EGFR genes. In an instance, when biomedical entities in the pair belong to the predefined classes: pathway and disease respectively, the biomedical entity belonging to the predefined class: disease may be caused due to an irregular event caused in the associated biomedical entity belonging to the predefined class: pathway. Referring to the first example, the biomedical entity “breast neoplasm” belonging to the predefined class: disease may be associated with a biomedical entity “mitogen-activated protein kinase (MAPK) signalling” belonging to the predefined class: pathway. Specifically, abnormal regulation of MAPK signalling pathway may be involved in the occurrence and progression of “breast neoplasm”. In yet another instance, when biomedical entities in the pair belong to the predefined classes: target and pathway respectively, the biomedical entity belonging to the predefined class: target may get affected because of an irregular or abnormal event in route of operation thereof. Such route of operation may be a biomedical entity belonging to the predefined class pathway. Furthermore, the association between the biomedical entity belonging to the predefined class: target and the biomedical entity belonging to the predefined class: pathway may be directly or indirect. Referring to the first example, the biomedical entity “EGFR” belonging to the predefined class: target may have an association with the biomedical entity “mitogen-activated protein kinase (MAPK) signalling” belonging to the predefined class: pathway. Specifically, overexpression of “EGFR” level in “MAPK signalling” pathway may cause occurrence and propagation of “breast neoplasm”.

Optionally, identifying at least one pair of biomedical entities, from the plurality of extracted biomedical entities, having an association therebetween comprises identifying potential associations between at least one pair of biomedical entities. Furthermore, potential associations refer to associations that are derived from existing associations. In an instance, a biomedical entity belonging to the predefined class: drug may be associated to a second biomedical entity belonging to the predefined class: disease. Additionally, a third biomedical entity belonging to the predefined class drug may be a potential drug for treating the second biomedical entity. Consequently, the second biomedical entity and the third biomedical entity may have a potential association therebetween. In an example, a first biomedical entity “Viagra”, belonging to a predefined class drug, may be used to treat a second biomedical entity “erectile dysfunction”, belonging to a predefined class disease, may have an association therebetween. Furthermore, the first biomedical entity “Viagra” may also be used in treating a third biomedical entity “pulmonary arterial hypertension”, belonging to the predefined class disease. Consequently, the first biomedical entity “Viagra” and the third biomedical entity “pulmonary arterial hypertension” may have a potential association therebetween. Additionally, optionally, the biomedical entities in each of the predefined classes may be scored based on an importance score thereof.

Optionally, potential associations between biomedical entities belonging to the predefined classes: drug and target, is identified by processing of data records associated with such biomedical entity. Specifically, for a biomedical entity belonging to the predefined class: drug, plurality of potential biomedical entities belonging to the predefined class: target is extracted from the data records. Consequently, drugability of each of the plurality of potential biomedical entities is analysed and the plurality of potential biomedical entities are filtered. Subsequently, the filtered plurality of potential biomedical entities are analysed based on properties associated therewith. Based on the properties associated with each of the plurality of potential biomedical entities belonging to the predefined class: target, potential association of the biomedical entity belonging to the predefined class: target is extracted with the biomedical entity belonging to the predefined class: target.

Optionally, the properties associated with each of the filtered plurality of biomedical entity belonging the predefined class: target, include likelihood of mutations, number of available gene expression studies and gene ontologies, and number of pathways associated with the biomedical entity. Specifically, the likelihood of mutations comprises the likelihood of genetic and somatic mutations in the biomedical entity. Furthermore, optionally, such identification of potential associations between biomedical entities belonging to the predefined classes: drug and target may be identified in an instance when either a target or drug is the input class.

Optionally, the method further comprises determining the importance score for each of the plurality of biomedical entities based on a first set of predetermined parameters. Furthermore, the processing module is operable to determine an importance score for each of the plurality of biomedical entities based on the first set of predetermined parameters. Specifically, importance factor of a biomedical entity may provide additional information regarding relevance of the biomedical entity with the user-input. Additionally, the importance score may further indicate researches and publications available associated to the biomedical entity. Furthermore, the importance score may also indicate most recent the biomedical entity in a predefined class. Optionally, the importance factor can be determined by applying a function on the first set of predetermined parameters. In an example, the first set of predetermined parameters may include number of PMID (PubMed Identifier), mechanism of action (MOA), number of associations of the biomedical entity and so forth. Furthermore, such first set of predetermined parameters may be different for biomedical entities in different predefined classes. In an instance, when the biomedical entity belongs to the predefined class: target, the first set of predetermined parameters for determining the importance score includes: a first subset of predetermined parameters and a second subset of predetermined parameters. The first subset of predetermined parameters includes: availability of approved drug for the target, availability of known structure of the target, presence of transmembrane helix, presence of genetic association and availability of gene expression data. The second subset of predetermined parameters includes number of PMID, number of antibodies, number of monoclonal antibodies, number of associated biomedical entities belonging to the predefined class pathways, number of associated biomedical entities belonging to the predefined classes drug and disease. Furthermore, the availability of approved drug for the target indicates availability of a known and tested drug for use on the target. Additionally, the availability of known structure of the biomedical entity indicates availability of information associated with anatomy of the biomedical entity. Moreover, the presence of transmembrane helix indicates presence of a membrane-spanning domain with a hydrogen-bonded helical configuration in the biomedical entity belonging to the predefined class target. Furthermore, the presence of genetic association indicates availability of information associated with genes and diseases related thereto with regard to the biomedical entity belonging to the predefined class target. Moreover, the availability of gene expression data relates to information associated with a process by which information from a gene is used in the synthesis of a functional gene product (such as, proteins, non-protein coding genes such as transfer RNA, small nuclear RNA). Specifically, a score of “1” (in case of presence of a parameter) or “0” (in case of absence of a parameter) is determined for each of the parameters in the first subset of parameters. Subsequently, an intermediate score may be calculated based on score of each of the parameters in the first subset of predetermined parameters. Furthermore, in the second subset of predetermined parameters, the number of PMID associated with the biomedical entity belonging to the predefined class: target indicates publications, literature and researches published with PubMed that have an association with the biomedical entity. Additionally, the number of antibody indicates count of proteins produced substantially by plasma cells that is used by immune system to neutralize pathogens such as bacteria and viruses. Moreover, the number of monoclonal antibody indicates number of antibodies that are made by identical immune cells that are all clones of a unique parent cell present in the biomedical entities belonging to the predefined class target. Furthermore, the importance score for the biomedical entity belonging to the predefined class target may be calculated based on each of: the intermediate score of the first subset of predetermined parameters, the second set of predetermined parameters. The importance score of the biomedical entity belonging to the predefined class target indicates drugability (ability to be treated) of the biomedical entity.

Optionally, in another instance, when the biomedical entity belongs to the predefined class: disease, the first set of predetermined parameters for determining the importance score includes: number of PMID, number of associations of the biomedical entity with biomedical entities of the predefined classes except the input class, number of grants specifying funding from government organizations such as World Health Organization, Department of Biotechnology and the like. Additionally, the importance factor may also depend on number of active and completed clinical trials associated with treatment of the biomedical entity belongs to the predefined class disease. Consequently, the aforementioned first set of predetermined parameters for determining the importance score for the biomedical entity belonging to the predefined class disease indicates cure and risk factors involved in treatment associated with the biomedical entity. Furthermore, the importance factor for the biomedical entity belonging to the predefined class disease indicates common or rare occurring of the biomedical entity in one or more subjects.

Optionally, in yet another instance, when the biomedical entity belongs to the predefined class: pathway, the first set of predetermined parameters for determining the importance score includes: number of PMID and number of associations with other biomedical entities belonging to the predefined classes except the input class. Furthermore, the number of PMID of such biomedical entity indicate publications and researches published with PubMed related to one or more disease and drugs associated with the biomedical entity belonging to the predefined class pathway. Additionally, the number of PMID of such biomedical entity may further indicate publications and researches related to mechanism of the biomedical entity. Moreover, the importance factor of the biomedical entity belonging to the predefined class pathway may indicate probability of association thereof with one or more diseases and drugs.

Optionally, in an instance when the biomedical entity belongs to the predefined class: drug, the first set of predetermined parameters for determining the importance score includes: number of PMID, number of associations with other biomedical entities belonging to the predefined classes except the input class, mechanism of action and pharmacokinetics. Specifically, PMID of the biomedical entity belonging to the predefined class: drug may indicate number of PubMed publications and researches related to the drug. Furthermore, number of associations of the biomedical entity with biomedical entities of predefined classes except the input class may indicate applicability of the biomedical entity. Furthermore, mechanism of action of the biomedical entity relates to process of working with associated biomedical entities. Moreover, pharmacokinetics may be used for determining the importance score by applying “Lipinski's rule of five elements”. Specifically, “Lipinski's rule of five elements” states that an orally active drug has no more than one violation in the following criteria: no more than five hydrogen bond donors, no more than ten hydrogen bond acceptors, a molecular mass less than 500 Daltons and an octanol-water partition coefficient log P not greater than five. Consequently, the aforementioned first set of predetermined parameters for determining the importance score for the biomedical entity belonging to the predefined class drug validate “drug likeness” (namely, ability to work as drug) of the biomedical entity belonging to the predefined class drug. Consequently, the importance score of a biomedical entity relates to aforementioned parameters associated to respective the biomedical entity. Specifically, the at least one pair of biomedical entities may have importance score related to each of the biomedical entities associated therein.

Optionally, the method further comprises determining a weightage score for each of the associations between the at least one pair of biomedical entities based on a second set of predefined parameters. Furthermore, the processing module is operable to determine the weightage score for each of the association between the at least one pair of biomedical entities based on the second set of predefined parameters. Specifically, a high weightage score indicates a well-known and approved association between the at least one pair of biomedical entities. Additionally, optionally, a low weightage score indicates a potential association. Furthermore, weightage score may affect representation scheme of the association between the at least one pair of biomedical entities based on a second set of predefined parameters. Moreover, the second set of predefined parameters may be different for each of the association between the at least one pair of biomedical entities. In an instance, when the biomedical entities in at least one pair belong to the predefined classes: drug and target, the second set of predefined parameters includes: hypergeometric association score based on PMID, assay count, animal model and well-known associations between the biomedical entities belonging to the predefined classes: drug and target. Furthermore, hypergeometric association score may be a method to identifying closest and known associations using hypergeometric distribution of the biomedical entities belonging to the predefined classes: drug and target based on number of PubMed publications thereof. Moreover, assay count refers to clinical trial count of the biomedical entity belonging to the predefined class: drug and testing thereof on the associated biomedical entity belonging to the predefined class drug. Furthermore, the animal model indicates that the biomedical entity belonging to the predefined class drug has been tested on the biomedical entity belonging to the predefined class target in an animal body. Moreover, the parameter well-known association between the biomedical entities belonging to the predefined classes: drug and target may act as a punishing score for the weightage score of the association thereof.

Optionally, in another instance when the at least one pair of biomedical entities in the association may belong to the predefined classes: drug and disease respectively. Additionally, at such an instance the second set of predefined parameters includes: hypergeometric association score based on the targets, clinical trials and well-known associations between the biomedical entities belonging to the predefined classes: drug and disease. Furthermore, hypergeometric association score may be a method to identifying closest and known associations between biomedical entities belonging to the predefined class: drug and disease using hypergeometric distribution of the biomedical entities based on association thereof with biomedical entities belonging to the predefined class: target. Moreover, number of completed and ongoing clinical trials for biomedical entity belonging to the predefined class: drug related to the biomedical entity belonging to the predefined class: disease. Furthermore, well-known association between biomedical entities belonging to the predefined classes: drug and disease may act as punishing score for the weightage score of the association.

Optionally, in another instance, when the at least one pair of biomedical entities in the association may belong to the predefined classes: target and disease respectively. Additionally, at such an instance the second set of predefined parameters includes: hypergeometric association score based on PMID, and well-known associations between the biomedical entities belonging to the predefined classes: target and disease. Specifically, hypergeometric association score may be a method to identifying closest and known associations using hypergeometric distribution of the biomedical entities belonging to the predefined classes: target and disease based on number of PubMed publications thereof. Moreover, availability of well-known associations between the biomedical entities belonging to the predefined classes: target and disease may act as punishing score for the weightage score of the association thereof.

Optionally, in an instance, when the at least one pair of biomedical entities in the association may belong to the predefined classes: pathway and disease respectively. Additionally, at such an instance the second set of predefined parameters includes: hypergeometric association score based on the targets, well-known associations between the biomedical entities belonging to the predefined classes: pathway and disease. Furthermore, hypergeometric association score may be a method to identifying closest and known associations between biomedical entities belonging to the predefined class: pathway and disease, using hypergeometric distribution of the biomedical entities based on association thereof with biomedical entities belonging to the predefined class: target. Additionally, well-known associations between the biomedical entities belonging to the predefined classes: pathway and disease may act as punishing score for the weightage score of the association thereof.

Optionally, in another instance, when the at least one pair of biomedical entities in the association may belong to the predefined classes: target and pathway. Additionally, at such an instance the second set of predefined parameters includes: hypergeometric association score based on PMID, and well-known associations between the biomedical entities belonging to the predefined classes: target and pathway. Specifically, hypergeometric association score may be a method to identifying closest and known associations using hypergeometric distribution of the biomedical entities belonging to the predefined classes: target and pathway based on number of PubMed publications thereof. Moreover, availability of well-known associations between the biomedical entities belonging to the predefined classes: target and pathway may act as punishing score for the weightage score of the association thereof. Additionally, punishing score used herein refers to a negative score that reduces the related weightage score when association thereof may be well-known. Beneficially, by way of punishing score, less-known associations between the at least one pair of biomedical entities are emphasized.

Consequently, the extracted plurality of biomedical entities related to the user-input and belonging to the predefined classes except the input class are paired based on association thereof. Subsequently, the paired biomedical entities belonging to the predefined classes except the input class are associated with the biomedical entity of the user-input.

As mentioned previously, the method further comprises: mapping the plurality of biomedical entities to the biomedical entity of the user-input. Specifically, the processing module is operable to map the plurality of biomedical entities to the biomedical entity of the user-input. Furthermore, the plurality of biomedical entities is paired and subsequently mapped to the biomedical entity user-input. Consequently, a mapped association of biomedical entities belonging to each of the predefined classes is determined. At an instance when the biomedical entity of the user-input belongs to the predefined class target, the biomedical entity of the user-input is mapped with the at least one pair of biomedical entity belonging to the predefined classes: disease, pathway and drug. At another instance when the biomedical entity of the user-input belongs to the predefined class disease, the biomedical entity of the user-input is mapped with the at least one pair of biomedical entity belonging to the predefined classes: target, pathway and drug. At yet another instance when the biomedical entity of the user-input belongs to the predefined class pathway, the biomedical entity of the user-input is mapped with the at least one pair of biomedical entity belonging to the predefined classes: target, disease and drug. At another instance when the biomedical entity of the user-input belongs to the predefined class drug, the biomedical entity of the user-input is mapped with the at least one pair of biomedical entity belonging to the predefined classes: target, disease and pathway. Furthermore, the mapped association of biomedical entities belonging to each of the predefined classes may be represented in form of a network, a tabular structure and so forth. Beneficially, the mapped association of biomedical entities belonging to each of the predefined classes may provide information related to biomedical entities belonging to any of the predefined classes, wherein the biomedical entities have an association therebetween.

Furthermore, there is disclosed a computer readable medium, containing program instructions for execution on a computer system, which when executed by a computer, cause the computer to perform method steps for mapping biomedical entities, wherein each of the biomedical entities belongs to one of a predefined class: target, disease, pathway, and drug. The method comprises the steps of providing a user-input of a biomedical entity belonging to one of the predefined classes, wherein the predefined class of the biomedical entity defines an input class; extracting a plurality of biomedical entities related to the biomedical entity of the user-input from existing data sources, wherein the plurality of biomedical entities belong to predefined classes except the input class; identifying at least one pair of biomedical entities, from the plurality of extracted biomedical entities, having an association therebetween, wherein each biomedical entity of the at least one pair of biomedical entities belongs to different predefined classes; and mapping the plurality of biomedical entities to the biomedical entity of the user-input.

Optionally, the computer readable medium comprises one of a floppy disk, a hard disk, a high capacity read only memory in the form of an optically read compact disk or CD-ROM, a DVD, a tape, a read only memory (ROM), and a random access memory (RAM).

### DETAILED DESCRIPTION OF THE DRAWINGS

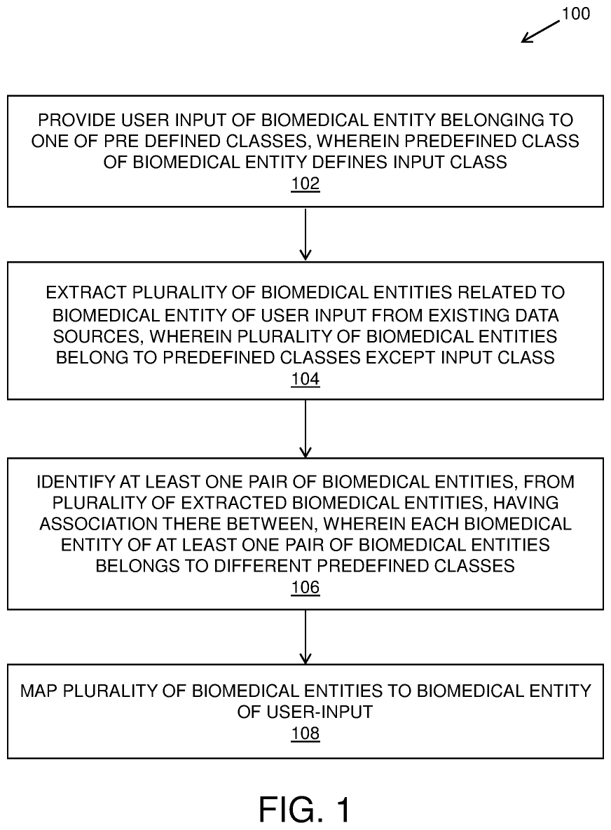

Referring to FIG. 1, illustrated are steps of a method 100 of mapping biomedical entities, in accordance with an embodiment of the present disclosure. Moreover, each of the biomedical entities belongs to one of a predefined class: target, drug, disease, and pathway. At a step 102, a user input of a biomedical entity belonging to one of the predefined classes is provided. Additionally, the predefined class of the biomedical entity defines an input class. At a step 104, plurality of biomedical entities related to the biomedical entity of the user input are extracted from existing data sources. Furthermore, the plurality of biomedical entities belongs to predefined classes except the input class. At a step 106, at least one pair of biomedical entities, having an association there between, is identified from the plurality of extracted biomedical entities. Moreover, each biomedical entity of the at least one pair of biomedical entities belongs to different predefined classes. At a step 108, the plurality of biomedical entities is mapped to the biomedical entity of the user-input.

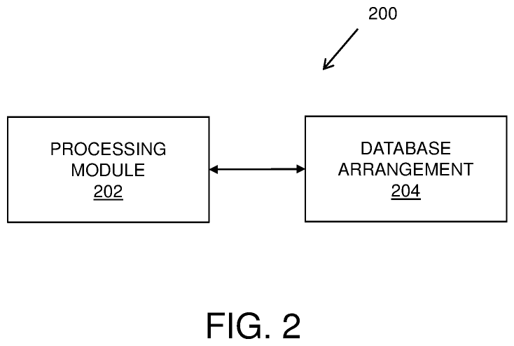

Referring to FIG. 2, illustrated is a block diagram of a system 200 that maps biomedical entities, in accordance with an embodiment of the present disclosure. The system 200 comprises a processing module 202 operable to map the biomedical entities. The processing module 200 is further operable to receive a user-input of a biomedical entity belonging to a predefined class. Subsequently, the processing module is operable to map plurality of biomedical entities to the biomedical entity of the user-input, wherein the plurality of biomedical entities has an association with the biomedical entity of the user-input. The system 200 further comprises a database arrangement 204 communicably coupled to the processing module 202. The database arrangement 204 is operable to store existing data sources.

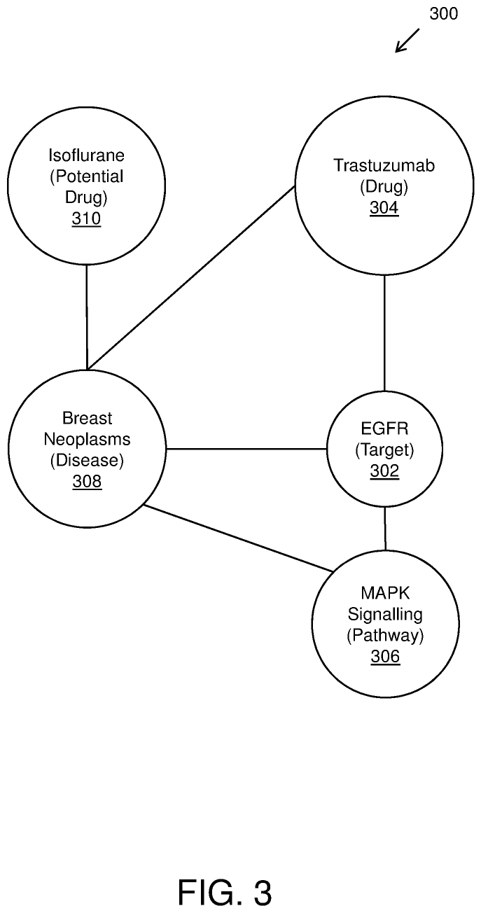

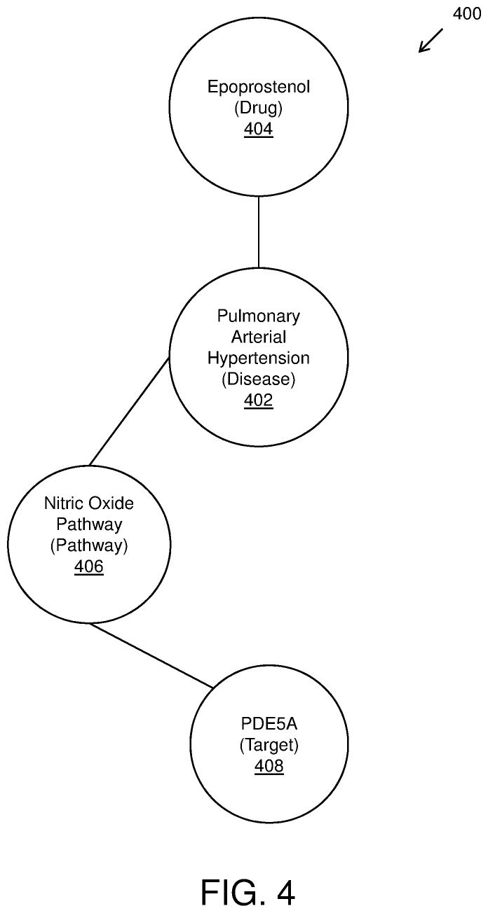

Referring to FIGS. 3 and 4, there are shown schematic illustrations of mapped associations of biomedical entities, in accordance with exemplary implementations of the present disclosure. The exemplary implementations relate to the method 100 (of FIG. 1).

In FIG. 3, a user input 302 of a biomedical entity “EGFR” belonging to the predefined class: target is provided. Consequently, a plurality of biomedical entities related to the biomedical entity of the user-input 302, such as biomedical entities “Trastuzumab” 304 belonging to the predefined class: drug, “MAPK signalling” 306 belonging to the predefined class: pathway, and “Breast Neoplasm” 308 belonging to the predefined class: disease, are extracted existing data sources. Subsequently, at least one pair of biomedical entities, such as the pair of biomedical entities 304 and 308, and 306 and 308, having an association therebetween is identified. Subsequently, the biomedical entities 304, 306, 308 are mapped to the biomedical entity of the user-input 302.

Optionally, the biomedical entity of user-input 302 acts as central element in mapped representation of biomedical entities. Optionally, a potential association may be identified between the biomedical entity “Breast Neoplasm” 308 belonging to the predefined class: disease and biomedical entity: Isoflurane 310 belonging to the predefined class: drug. Specifically, Isoflurane 310 is a potential drug associated to the biomedical entity Breast Neoplasm 308.

In FIG. 4, a user-input 402 of biomedical entity “Pulmonary Arterial Hypertension” belonging to the predefined class: disease, is provided. Therefore, in such a case, the input class is defined as the disease. Consequently, a plurality of biomedical entities related to the biomedical entity of the user-input 302, such as: Epoprostenol 404 belonging to the predefined class: drug and Nitric Oxide Pathway 406 belonging to the predefined class: pathway, are extracted. Furthermore, the biomedical entity Nitric Oxide Pathway 406 is associated to biomedical entity PDE5A 408 belonging to the predefined class: target.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

What is claimed is:

1. A system that maps biomedical entities, wherein each of the biomedical entities belongs to one of a predefined class: target, disease, pathway, and drug, wherein the system includes a computer system, wherein the system comprises:
a database arrangement operable to store existing data sources; and
a processing module communicably coupled to the database arrangement, the processing module operable to:
receive a user-input of a biomedical entity belonging to one of the predefined classes, wherein the predefined class of the biomedical entity defines an input class;
extract a plurality of biomedical entities related to the biomedical entity of the user-input from existing data sources, wherein the plurality of biomedical entities belong to predefined classes except the input class;
identify at least one pair of biomedical entities, from the plurality of extracted biomedical entities, having an association therebetween, wherein each biomedical entity of the at least one pair of biomedical entities belongs to different predefined classes; and
map the plurality of biomedical entities to the biomedical entity of the user-input.

2. The system of claim 1, wherein the processing module is operable to determine an importance score for each of the plurality of biomedical entities based on a first set of predetermined parameters.

3. The system of claim 1, wherein the processing module is operable to determine a weightage score for each of the association between the at least one pair of biomedical entities based on a second set of predefined parameters.

4. The system of claim 1, wherein identifying at least one pair of biomedical entities, from the plurality of extracted biomedical entities, having an association therebetween comprises identifying potential associations between at least one pair of biomedical entities.

5. The system of claim 1, wherein the at least one pair of biomedical entities having the association therebetween comprises biomedical entities belonging to at least one of the following different predefined classes: target and drug, drug and disease, target and disease, pathway and disease, target and pathway.

6. The system of claim 1, wherein the processing module is operable to tag the biomedical entities in the predefined class: drug, with one of the tags: approved, investigational, combinational, and potential.

7. A method of mapping biomedical entities, wherein each of the biomedical entities belong to one of a predefined class: target, disease, pathway, and drug, wherein the method includes using a computer system, wherein the method comprises:
providing a user-input of a biomedical entity belonging to one of the predefined classes, wherein the predefined class of the biomedical entity defines an input class;
extracting a plurality of biomedical entities related to the biomedical entity of the user-input from existing data sources, wherein the plurality of biomedical entities belongs to predefined classes except the input class;
identifying at least one pair of biomedical entities, from the plurality of extracted biomedical entities, having an association therebetween, wherein each biomedical entity of the at least one pair of biomedical entities belongs to different predefined classes; and
mapping the plurality of biomedical entities to the biomedical entity of the user-input.

8. The method of claim 7, wherein the method further comprises determining an importance score for each of the plurality of biomedical entities based on a first set of predetermined parameters.

9. The method of claim 7, wherein the method further comprises determining a weightage score for each of the association between the at least one pair of biomedical entities based on a second set of predefined parameters.

10. The method of claim 7, wherein identifying at least one pair of biomedical entities, from the plurality of extracted biomedical entities, having an association therebetween comprises identifying potential associations between at least one pair of biomedical entities.

11. The method of claim 7, wherein the at least one pair of biomedical entities having the association therebetween comprises biomedical entities belonging to at least one of the following different predefined classes: target and drug, drug and disease, target and disease, pathway and disease, target and pathway.

12. The method of claim 7, wherein the method further comprises tagging the biomedical entities in the predefined class: drug, with one of the tags: approved, investigational, combinational, and potential.

13. A computer readable medium, containing program instructions for execution on a computer system, which when executed by a computer, cause the computer to perform method steps for mapping biomedical entities, wherein each of the biomedical entities belongs to one of a predefined class: target, disease, pathway, and drug, the method comprising the steps of:
providing a user-input of a biomedical entity belonging to one of the predefined classes, wherein the predefined class of the biomedical entity defines an input class;
extracting a plurality of biomedical entities related to the biomedical entity of the user-input from existing data sources, wherein the plurality of biomedical entities belongs to predefined classes except the input class;
identifying at least one pair of biomedical entities, from the plurality of extracted biomedical entities, having an association therebetween, wherein each biomedical entity of the at least one pair of biomedical entities belongs to different predefined classes; and
mapping the plurality of biomedical entities to the biomedical entity of the user-input.
